SEQUENCE LISTING (1 sequence)

SEQ ID NO: 1 - protein, 333 aa, Homo sapiens
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

(19) United States
(12) Patent Application Publication
(10) Pub. No.: US 2022/0064285 A1
(43) Pub. Date: Mar. 3, 2022

(54) ANTI-EPHRIN-B2 BLOCKING ANTIBODIES FOR THE TREATMENT OF FIBROTIC DISEASES

(71) Applicant: The General Hospital Corporation, Boston, MA (US)

(72) Inventor: David Lagares, Somerville, MA (US)

(21) Appl. No.: 17/418,343

(22) PCT Filed: Dec. 27, 2019
(86) PCT No.: PCT/US2019/068746

§ 371 Date: Jun. 25, 2021

Related U.S. Application Data
(60) Provisional application No. 62/785,873, filed on Dec. 28, 2018.

(51) Int. Cl. C07K 16/28; A61P 11/00

(52) U.S. Cl. CPC C07K 16/28; C07K 2317/24; C07K 2317/76; A61P 11/00

(57) ABSTRACT

Methods and compositions for treating organ fibrosis using antibodies or antigen-binding fragments thereof that bind to and block the soluble Ephrin B2 ectodomain.

### CLAIM OF PRIORITY

This application claims the benefit of U.S. Provisional Patent Application Ser. No. 62/785,873, filed on Dec. 28, 2018. The entire contents of the foregoing are hereby incorporated by reference.

### TECHNICAL FIELD

Described herein are methods and compositions for treating organ fibrosis using antibodies or antigen-binding fragments thereof that bind to and block the soluble Ephrin-B2 (sEphrin-B2) ectodomain.

### BACKGROUND

The ability of organs to regenerate following injury declines with age. In aged individuals, chronic tissue injury leads to abnormal wound healing responses characterized by the development of scar tissue or fibrosis and subsequent organ failure.

### SUMMARY

Maladaptive wound healing responses to chronic tissue injury result in organ fibrosis. Fibrosis, which entails excessive extracellular matrix (ECM) deposition and tissue remodeling by activated myofibroblasts, leads to loss of proper tissue architecture and organ function. The ADAM10-sEphrin-B2 pathway is a major driver of myofibroblast activation¹⁶. As shown herein, in addition to being involved in the development of fibrosis, this pathway can be specifically targeted for therapeutic intervention in subjects diagnosed with fibrosis, in particular using strategies to block sEphrin-B2 directly using neutralizing antibodies. Thus anti-ephrin-B2 antibodies can be used to treat lung fibrosis in patients with fibrosis, e.g., Idiopathic Pulmonary Fibrosis (IPF). At the time of diagnosis, lung fibrosis is by definition established but more importantly progressive. Anti-ephrin-B2 antibodies can be used to treat progressive lung fibrosis, including early and late disease, as well as fibrosis present in other organs (e.g., systemic fibrosis/scleroderma, or liver fibrosis or cirrhosis, among others).

Thus provided herein are methods for treating organ fibrosis in a subject. The methods include identifying a subject who has organ fibrosis, and administering a therapeutically effective amount of one or more antibodies or antigen binding fragments thereof that bind to and block soluble ephrin-B2 ectodomain.

In some embodiments, the organ fibrosis is pulmonary (e.g., idiopathic pulmonary fibrosis), skin, kidney fibrosis, liver fibrosis or cirrhosis, systemic sclerosis, or desmoplastic tumors.

In some embodiments, the treatment results in a reduction in fibrosis and/or a return or approach to normal function of the organ.

In some embodiments, the subject has pulmonary fibrosis, and the therapeutically effective amount results in decreased lung fibrosis and improved lung function, e.g., improved oxygenation and/or normalization of forced vital capacity (FVC).

In some embodiments, the subject has pulmonary fibrosis, e.g., has patterns of fibrosis on a chest radiograph or chest computed tomography (CT) or high-resolution CT (HRCT) scan, and bibasilar inspiratory crackles.

In some embodiments, the subject has systemic sclerosis (SSc), e.g., has skin thickening of the fingers, finger tip lesions, telangiectasia, abnormal nailfold capillaries, interstitial lung disease or pulmonary arterial hypertension, Raynaud's phenomenon, and SSc-related autoantibodies.

In some embodiments, the subject has liver fibrosis or cirrhosis, e.g., has fibrosis detected on biopsy or imaging, e.g., on ultrasound (US), computed tomography (CT), Fibroscan, or MR imaging (MRI).

In some embodiments, the antibody is a clone B11 or 2B1 antibody.

In some embodiments, the antibody is a monoclonal chimeric, de-immunized or humanized antibody.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-B. Ephrin-B2 is upregulated in IPF fibroblasts. (A,B) mRNA and protein expression levels of ephrin-B2 in human lung fibroblasts from healthy control donors (n=3) and individuals with IPF (n=3).

FIGS. 2A-C. Fibroblast-specific Ephrin-B2 KO mice are protected from bleomycin-induced lung fibrosis. (A) Masson's trichrome staining of lung sections from control wild type (WT) and fibroblast-specific Ephrin-B2 KO mice 14 d after PBS or bleomycin (BLM) challenge. (B) Hydroxyproline content in WT (left hand bars) and KO (right hand). (C) α-SMA and type I collagen protein expression. n=6 mice for all groups.

FIGS. 3A-C. Ephrin-B2 ectodomain is shed into the alveolar space upon lung injury. (A) Western blot showing ephrin-B2 expression levels in total lung homogenates from WT mice harvested at 14 d following PBS or bleomycin challenge. The arrow indicates the appearance of the lower-molecular-weight band (˜50 kDa). (B) Western blot showing cleaved sEphrin-B2 levels in BAL fluids from PBS- (left bars) and bleomycin (right bars)-challenged mice at day 14 after treatment (C) Concentration of sEphrin-B2, as determined by ephrin-B2 ELISA in BAL fluid from WT mice at different time points in the model.

FIGS. 4A-F. The soluble ephrin-B2 ectodomain is sufficient to drive myofibroblast formation and tissue fibrosis. (A) Domain structure of the full length ephrin-B2 protein and the recombinant soluble ephrin-B2 ectodomain fused to Fc. (B) Effects of Ephrin-B2-Fc or IgG-Fc control on α-SMA and type I collagen protein expression in mouse lung fibroblasts. (C) WT mice were treated with daily subcutaneous injections of either pre-clustered Ephrin-B2-Fc (n=6) or IgG-Fc (n=6) as control for 14 d. H&E and Masson's trichrome staining are shown. (D-F) Quantification of dermal fibrosis markers, dermal thickness (D) and hydroxyproline (E) (left bars, IgG-Fc; right bars, Ephrin-B2-Fc), and western blot showing α-SMA and type I collagen levels in skin (F).

FIG. 5. Effect of control (left bars) or anti-Ephrin-B2 neutralizing antibody (B11, right bars) on TGF-β-induced α-SMA expression in lung fibroblasts.

FIG. 6. Treatment strategies with anti-ephrin-B2 blocking antibody. WT mice will be treated with bleomycin or saline via intratracheal instillation and anti-ephrin-B2 blocking antibody or control antibody as indicated.

FIGS. 7A-C. Anti-ephrin-B2 antibody prevents myofibroblast activation and bleomycin-induced lung fibrosis in mice. (A) Representative images (from n=6 mice per group) of Masson's trichrome staining (collagen type I) of lung sections from mice at 21 d after PBS or bleomycin challenge that were treated with anti-ephrin-B2 antibody (B11 clone) or control IgG2a antibody. (B) Hydroxyproline content (collagen levels) measured in the lungs of mice (n=6 for all groups). (C) α-SMA mRNA expression assessed by real time PCR in total lung homogenates (n=6 for all groups).

FIGS. 8A-C. Anti-ephrin-B2 antibody prevents myofibroblast activation driven by TGF-β and the fibrotic phenotype of lung fibroblasts from patients with IPF. (A) Concentration of sEphrin-B2 as determined by ELISA in conditioned media from primary lung fibroblasts from control (healthy) donors (n=5) and individuals with IPF (n=5). (B) Effect of anti-ephrin-B2 antibody (B11 clone) on TGF-β-induced α-SMA protein expression on primary lung fibroblasts from control donors (n=3), with GAPDH used as a loading control. n=3 for all groups. Anti-ephrin-B2 antibody prevents TGF-β-induced α-SMA protein expression (P<0.05). (C) Effect of anti-ephrin-B2 antibodies (B11 and 2B1 clones) on α-SMA protein expression and phospho-SMAD3 on primary lung fibroblasts from control donors (n=3) and individuals with IPF (n=3), with GAPDH used as a loading control. n=3 for all groups.

FIGS. 9A-B. sEphrin-B2 levels are upregulated in BAL and plasma from IPF patients. (A) Concentration of sEphrin-B2 in BAL fluid (A) and plasma (B) from control donors (n=30) and IPF patients (n=30) assessed by ELISA. **P<0.01

FIG. 10. sEphrin-B2 levels correlates with clinical outcome of IPF patients. Increased plasma sEphrin-B2 associates with increased mortality in patients with IPF (n=30, *P<0.03)

### DETAILED DESCRIPTION

The identification of novel therapeutic strategies aiming at reducing tissue fibrosis and promoting the regeneration of damaged tissues is a major unmet clinical need in regenerative medicine. The present disclosure uncovers a new molecular mechanism of tissue fibrogenesis and demonstrates that targeting the ADAM10-soluble Ephrin-B2 pathway in scar-forming myofibroblasts reverses established lung fibrosis and restores organ function. The present findings reveal novel therapeutic targets for the treatment of a variety of human fibrotic diseases such as idiopathic pulmonary fibrosis, systemic sclerosis (scleroderma), liver cirrhosis, kidney fibrosis and desmoplastic tumors.

Targeting the ADAM10-sEphrin-B2 pathway in lung fibrosis. Chronic lung diseases are among the leading causes of death in the United States. Idiopathic Pulmonary Fibrosis (IPF) is a common lung disease that invariably leads to a progressive decline in lung function, resulting in significant morbidity and mortality¹⁻³. Patients with IPF suffer from irreversible and ultimately fatal interstitial lung disease characterized by progressive lung scarring (fibrosis), ultimately impeding the ability to breath^{4,5}. Recent epidemiologic studies suggest that IPF affects more persons than previously appreciated⁶⁻⁸. The prevalence of IPF in the U.S. has recently been estimated to range from 10-60 cases per 100,000 persons⁶, indicating that there may be as many as 130,000 persons in the U.S. with diagnosed IPF, and as many as 34,000 persons developing IPF each year⁹. The prognosis of IPF is poor. The median survival is between 2 and 5 years from time of diagnosis¹⁰. Current therapy mainly relies on two recently licensed anti-fibrotic drugs (pirfenidone and nintedanib) or symptomatic treatments that modestly slow the decline in lung function in some IPF patients^{11,12}, but cannot halt or reverse the disease progression.

IPF is associated with unacceptably high morbidity and mortality. The development of more effective therapies will require improved understanding of the biological processes involved in the pathogenesis of pulmonary fibrosis, and more complete identification of the molecular mediators regulating these processes. Activation of scar-forming myofibroblasts is a critical step in the progressive scarring that underlies the development and progression of pulmonary fibrosis^{3,13}. Myofibroblasts demonstrate increased collagen synthesis and expression of α-smooth muscle actin (α-SMA), which confers them a hyper-contractile phenotype to remodel the ECM¹⁴. Consequently, targeting molecular pathways responsible for myofibroblast activation has therefore great potential as a treatment strategy for IPF^{3,13-15}.

The ADAM10-sEphrin-B2 pathway was recently identified as a major driver of myofibroblast activation in patients with IPF and in mouse models of lung fibrosis¹⁶. Ephrin-B2 is a transmembrane ligand highly expressed in quiescent lung fibroblasts^{16,17}, however its pro-fibrotic effects are regulated by an activation step that occurs upon lung injury. Recent studies have demonstrated that following lung injury the ectodomain of full-length ephrin-B2 in quiescent lung fibroblasts is proteolytically cleaved by the disintegrin and metalloproteinase ADAM10, resulting in the generation of the biologically active molecule soluble Ephrin-B2 (sEphrin-B2). Once shed, sEphrin-B2 generates pro-fibrotic signaling to quiescent fibroblasts by activating EphB4 receptor signaling in an autocrine/paracrine manner. The present studies demonstrate that sEphrin-B2/EphB4 receptor signaling promotes differentiation of quiescent fibroblasts into activated myofibroblasts and is sufficient to drive tissue fibrosis in mice. Moreover, mice genetically lacking ephrin-B2 specifically in lung fibroblasts exhibit significant protection from bleomycin-induced lung fibrosis. Surprisingly, administration of anti-sEphrin-B2 antibodies reverses established fibrosis. Consequently, strategies to interrupt the elaboration of sEphrin-B2, by blocking sEphrin-B2 directly, serve as novel therapeutic strategies for fibrosis.

Methods of Treatment

As demonstrated herein, soluble ephrin-B2 is sufficient to drive activation of scar-forming myofibroblasts in the lungs and skin. It is thought that pathological mechanisms involved in myofibroblasts activation are conserved across organs (see, e.g., Rockey et al., N Engl J Med. 2015 Mar. 19; 372(12):1138-49). Thus, targeting pathways involved in maintaining the fibrogenic state of myofibroblasts represent pan anti-fibrotic targets for fibrotic disorders. See also Zeisberg and Kalluri, Am J Physiol Cell Physiol. 2013 Feb. 1; 304(3):C216-25.

The methods described herein include methods for treating organ fibrosis, e.g., pulmonary (e.g., idiopathic pulmonary fibrosis), skin, kidney fibrosis, liver fibrosis or cirrhosis, systemic sclerosis, and desmoplastic tumors. Generally, the methods include administering a therapeutically effective amount of antibodies that bind to and block soluble ephrin-B2 ectodomain as described herein, to a subject who is in need of, or who has been determined to be in need of, such treatment. The antibodies can be neutralizing antibodies (e.g., clone B11) and/or those that sterically hinder the binding of soluble ephrin B2 (e.g., clone 2B1).

As used in this context, to “treat” means to ameliorate at least one symptom of the organ fibrosis. Often, organ fibrosis results in scarring and thickening of the tissue, and a loss of or reduction in function; thus, a treatment can result in a reduction in fibrosis and a return or approach to normal function of the organ. For example, administration of a therapeutically effective amount of a compound described herein for the treatment of a condition associated with pulmonary will result in decreased lung fibrosis and improved lung function, e.g., improved oxygenation and/or normalization of forced vital capacity (FVC).

The methods can be used in any subject who has organ fibrosis. Methods for identifying or diagnosing subjects who have organ fibrosis are known in the art; see, e.g., Raghu et al., Am J Respir Crit Care Med. 2018 Sep. 1; 198(5):e44-e68 and Martinez et al., Lancet Respir Med. 2017 January; 5(1):61-71 for IPF; van den Hoogen et al., Arthritis Rheum. 2013 November; 65(11):2737-47 for scleroderma; and Lurie et al., World J Gastroenterol. 2015 Nov. 7; 21(41):11567-83 and Li et al., Cancer Biol Med. 2018 May; 15(2): 124-136 for liver fibrosis and cirrhosis.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. It can also refer to a sufficient amount of a anti-Ephrin B2 antibody to retard, delay or reduce the risk of progression of a disease or condition, symptoms associated with a disease or condition or otherwise result in an improvement in an accepted characteristic of a disease or condition when administered according to a given treatment protocol. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

sEphrin B2 (sEphrinB2) Antibodies—Pharmaceutical Compositions and Methods of Administration

Ephrin-B2 binding to EphB receptors is mediated by highly conserved surface regions in the ephrin-B2 ectodomain, whose crystal structure has been recently resolved (Toth et al., Dev Cell. 2001 July; 1(1):83-92; Qin et al., J Biol Chem. 2010 Jan. 1; 285(1):644-54; Himanen et al., Nature. 2001 Dec. 20-27; 414(6866):933-8). We have found that the ephrin-B2 ectodomain required to activate EphB receptors is shed upon lung injury, and that soluble ectodomain is biologically active and capable of binding and activating EphB receptor signaling in lung fibroblasts. Because EphB receptor activation by sEphrin-B2 ectodomain induces myofibroblast activation, hampering this protein-protein interaction could have potential medical applications as anti-fibrotic therapy for the treatment of organ fibrosis. One approach to inhibit sEphrin-B2 signaling is to use blocking antibodies against ephrin-B2 ectodomain, which neutralize its binding and activation of EphB receptors (i.e., EphB3 and EphB4). Highly specific ephrin-B2 blocking antibodies that both bind the ectodomain and prevent receptor signaling have been developed²⁶. Thus the present methods can include administration of compositions comprising a therapeutically effective amount of an antibody, or an antigen-binding portion thereof, that binds to the EphrinB2 ectodomain and prevent receptor signaling.

The methods described herein include the use of pharmaceutical compositions comprising sEphrin-B2 antibodies as an active ingredient. The term “antibody” as used herein refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments the antibody has effector function and can fix complement. The antibodies or fragments of the antibodies can be treated to include any of the post-translational modifications that are known in the art and commonly applied to antibodies, provided that the modified antibodies or fragments maintain specificity for binding to human or murine Ephrin B2. Modifications may include PEGylation, phosphorylation, methylation, acetylation, ubiquitination, nitrosylation, glycosylation, ADP-ribosylation, or lipidation. Alternatively, or in addition, the antibodies or fragments may further comprise a detectable label that can be used to detect binding in an immunoassay. Labels that may be used include radioactive labels, fluorophores, chemiluminescent labels, enzymatic labels (e.g., alkaline phosphatase or horseradish peroxidase); biotin; avidin; and heavy metals. In some embodiments, the antibody has reduced or no ability to bind an Fc receptor. For example, the antibody can be an isotype or subtype, fragment or other mutant, which does not support binding to an Fc receptor, e.g., it has a mutagenized or deleted Fc receptor binding region. In addition to the sEphrinB2 antibodies described above, other antibodies can be made. Methods for making antibodies and fragments thereof are known in the art, see, e.g., Harlow et. al., editors, Antibodies: A Laboratory Manual (1988); Goding, Monoclonal Antibodies: Principles and Practice, (N.Y. Academic Press 1983); Howard and Kaser, Making and Using Antibodies: A Practical Handbook (CRC Press; 1st edition, Dec. 13, 2006); Kontermann and Dübel, Antibody Engineering Volume 1 (Springer Protocols) (Springer; 2nd ed., May 21, 2010); Lo, Antibody Engineering: Methods and Protocols (Methods in Molecular Biology) (Humana Press; Nov. 10, 2010); and Dübel, Handbook of Therapeutic Antibodies: Technologies, Emerging Developments and Approved Therapeutics, (Wiley-VCH; 1 edition Sep. 7, 2010). The sequence of human EphrinB2 is provided in GenBank at Acc No. NM_004093.3 (nucleic acid) and NP_004084.1 (protein). An exemplary sequence of full length human EphrinB2 precursor is as follows:

```
(SEQ ID NO: 1)
    1 MAVRRDSVWK YCWGVLMVLC RTAISKSIVL EPIYWNSSNS KFLPGQGLVL
        YPQIGDKLDI
  61 ICPKVDSKTV GQYEYYKVYM VDKDQADRCT IKKENTPLLN CAKPDQDIKF
        TIKFQEFSPN
121 LWGLEFQKNK DYYIISTSNG SLEGLDNQEG GVCQTRAMKI LMKVGQDASS
        AGSTRNKDPT
181 RRPELEAGTN GRSSTTSPFV KPNPGSSTDG NSAGHSGNNI LGSEVALFAG
        IASGCIIFIV
241 IIITLVVLLL KYRRRHRKHS PQHTTTLSLS TLATPKRSGN NNGSEPSDII
        IPLRTADSVF
301 CPHYEKVSGD YGHPVYIVQE MPPQSPANIY YKV.
```

In some embodiments, the EphrinB2 Ectodomain comprises or consists of amino acids 29 to 165 of SEQ ID NO:1 (bold font above).

Pharmaceutical compositions typically include a pharmaceutically acceptable carrier. As used herein the language “pharmaceutically acceptable carrier” includes diluent, saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral, e.g., intravenous, intradermal, subcutaneous, intraperitoneal, oral (e.g., inhalation, intranasal), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration. Thus also included herein are devices, such as inhalers, that comprise an sEphrinB2 antibody, e.g., for use in a method described herein.

### EXAMPLES

The invention is further described in the following examples, which do not limit the scope of the invention described in the claims.

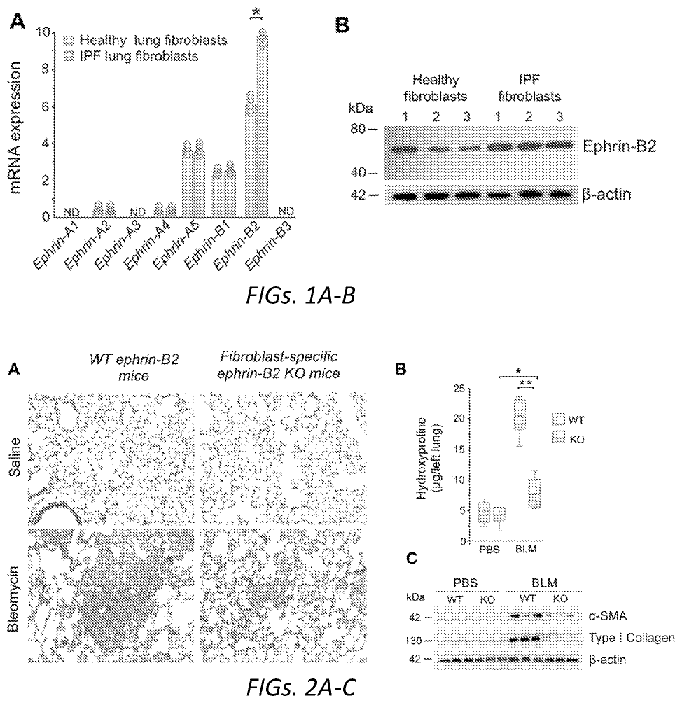

Ephrin-B2 is upregulated in IPF fibroblasts. We have previously shown increased expression of genes associated with migration and activation of fibroblasts in the lungs of patients with rapidly progressive IPF¹⁸. To identify putative genes that regulate activation and migration in IPF fibroblasts, we analyzed publicly available microarray data sets comparing the gene expression of lung fibroblasts isolated from individuals with IPF to that of healthy lung fibroblasts used as controls^{19,20}, and found EFNB2, the gene encoding the transmembrane protein ephrin-B2 was significantly increased in the IPF lung fibroblasts. EFNB2 (Gene Expression Omnibus accession number GSE1724)¹⁹ encodes the transmembrane protein ephrin-B2, which belongs to the family of ephrin ligands which bind to Eph receptors at the surface of adjacent cells²¹. The ephrin family of ligands is divided by structure into phosphatidylinositol-linked ephrin-A ligands (ephrin-A1-6) and transmembrane ephrin-B ligands (ephrin-B1-3)^{17,21}. Both ephrin-A and -B ligands bind to Eph receptors at the surface of adjacent cells to initiate biochemical signaling'. Among all the ephrin-A and -B ligands, ephrin-B2 is the highest ephrin ligand expressed in lung fibroblasts with its expression upregulated in lung fibroblasts from patients with IPF¹⁹. We also confirmed that expression of ephrin-B2, but not other members of the ephrin family of ligands, is markedly higher in lung IPF fibroblasts compared with lung fibroblasts isolated from control subjects, as demonstrated by mRNA and protein analyses (FIG. 1A,B).

Fibroblast-specific ephrin-B2-deficient mice are protected from bleomycin-induced lung fibrosis. We then investigated whether fibroblast ephrin-B2 is required for the development of fibrosis in vivo. As mice that are globally ephrin-B2-deficient die at mid-gestation owing to defective cardiovascular development, we generated mice in which we could conditionally delete Efnb2 in collagen-expressing cells, such as fibroblasts. We crossed mice with Efnb2 flanked by loxP sites (Efnb2loxP/loxP mice) to mice that express a tamoxifen-inducible Cre recombinase driven by the mouse promoter of Colla2 (collagen, type I, alpha 2) (Colla2-CreERT mice). Tamoxifen treatment of offspring that were homozygous for the ‘foxed’ Efnb2 allele and hemizygous for the Colla2-Cre transgene (Efnb2loxP/loxP; Colla2-CreERT mice), as confirmed by PCR, led to the deletion of the Efnb2 gene in fibroblasts and the generation of Efnb2 conditional knockout mice. Littermates treated with corn oil vehicle alone were used as controls. Western blotting for ephrin-B2 protein demonstrated markedly lower expression in extracts from lung fibroblasts of Ephrin-B2 KO mice compared to control mice. Our studies demonstrated that fibroblast-specific ephrin-B2-deficient mice showed marked protection from the development of lung fibrosis induced by bleomycin compared to wild type (WT) mice as demonstrated by histological (Masson's trichrome stain for collagen accumulation), biochemical (hydroxyproline level for collagen content) and molecular (type I collagen and α-SMA, a marker of myofibroblast differentiation) assessments (FIG. 2A-C).

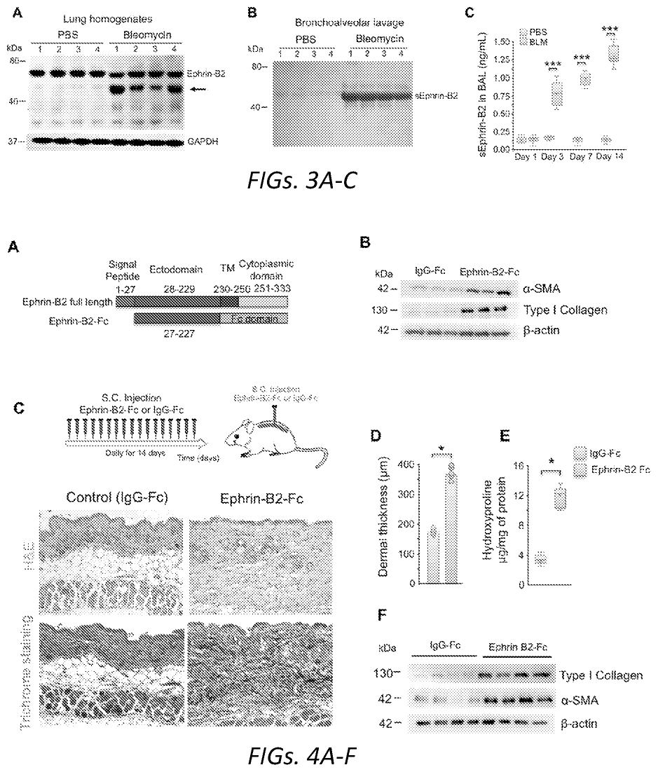

Ephrin-B2 ectodomain is shed upon lung injury. We found that bleomycin challenge did not increase expression of the full-length transmembrane ephrin-B2 (˜60 kDa) but resulted in the generation of a lower-molecular-weight band (˜50 kDa) that was absent in control lungs (FIG. 3A, arrow indicates the 50 kDa band). Since ephrin-B ligands have been shown to undergo ectodomain shedding to release active proteins²²⁻²⁵, we hypothesized that proteolytic cleavage of ephrin-B2 following bleomycin injury resulted in the generation of a 50-kDa soluble form of ephrin-B2, herein referred to as sEphrin-B2, which could contribute to the pathogenesis of lung fibrosis. Our results demonstrated significant increases in sEphrin-B2 in cell-free supernatants from the bronchoalveolar lavage (BAL) fluid of WT mice following bleomycin challenge as demonstrated by Western Blotting and by enzyme-linked immunosorbent assay (ELISA) using an ectodomain-specific anti-ephrin-B2 monoclonal antibody (FIG. 3B,C). Together, our results demonstrated that ephrin-B2 ectodomain is shed following lung injury.

Soluble ephrin-B2 ectodomain is sufficient to drive myofibroblast activation and tissue fibrosis. To test whether sEphrin-B2 functioned directly as a profibrotic mediator, we treated fibroblasts with a recombinant ephrin-B2 ectodomain-Fc, which contains the ectodomain of ephrin-B2 fused to an Fc domain that replaces the transmembrane and C-terminal domains of the full-length ephrin-B2 protein (FIG. 4A). Treatment of primary mouse lung fibroblasts with preclustered ephrin-B2-Fc markedly increased α-SMA and type I collagen protein expression compared to control IgG-Fc treatment, supporting a direct pro-fibrotic effect (FIG. 4B). Next, we administered pre-clustered ephrin-B2-Fc (100 μg/kg) or control IgG-Fc subcutaneously daily for 2 weeks and identified a marked increase in dermal fibrosis as assessed by increased dermal thickness, hydroxyproline content and α-SMA and type I collagen expression compared control (FIG. 4C-F). Together, these data show that the ephrin-B2 ectodomain is sufficient to drive myofibroblast activation and tissue fibrosis in vivo.

Therapeutic antibodies against sEphrin-B2 for the treatment of lung fibrosis in IPF. Our studies demonstrate that subcutaneous injection of sEphrin-B2 ectodomain is sufficient to induce tissue fibrosis in mice in vivo by inducing myofibroblast activation. Together, our results suggest that therapeutic inhibition of sEphrin-B2 signaling could represent a novel strategy to mitigate lung fibrosis by preventing myofibroblast activation. Therapeutic strategies aiming at blocking ephrin-B2 signaling have been previously developed for cancer treatment^{26,27}, however their anti-fibrotic effects have been never explored.

Because EphB4 receptor activation by sEphrin-B2 ectodomain induces myofibroblast activation, hampering this protein-protein interaction could have potential medical applications as anti-fibrotic therapy for the treatment of IPF. One approach to inhibit sEphrin-B2 signaling is to develop blocking antibodies against ephrin-B2 ectodomain, which would neutralize its binding and activation of EphB4 receptor. Using a human antibody phage display library, a potent anti-ephrin-B2 antibody (clone B11) was identified that neutralizes ephrin-B2 binding to EphB4 receptor²⁶. Recent studies have validates B11 as a potent research tool in preclinical models of melanoma and breast cancer ^{26,27as} well as xenograft models²⁶.

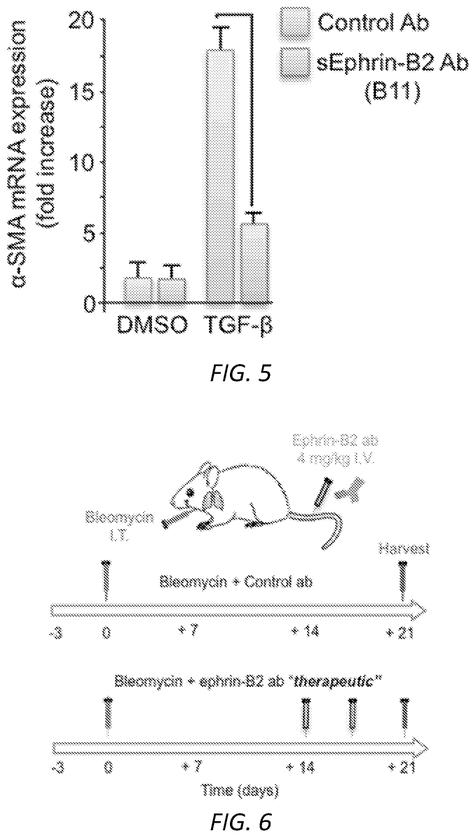

Blockade of sEphrin-B2 with the B11 neutralizing antibody prevents TGF-β-induced myofibroblast formation. In order to investigate the therapeutic efficacy of sEphrin-B2 blocking antibodies in vitro, we assessed myofibroblast activation by α-SMA expression in primary human lung fibroblast treated with TGF-β in the presence or absence of B11 anti-ephrin-B2 blocking antibody (100 μg/mL=3 μM) for 48 hours. As shown in FIG. 5, B11 pre-treatment significantly reduces TGF-β-induced α-SMA expression in lung fibroblasts.

Blockade of sEphrin-B2 with the B11 neutralizing antibody reverses lung fibrosis in mouse models. On the basis of our findings above, we hypothesized that therapeutic inhibition of sEphrin-B2 with neutralizing antibodies could treat lung fibrosis by inhibiting myofibroblast activation in vivo. In order to investigate the therapeutic efficacy of sEphrin-B2 blocking antibodies in vivo, we investigated the ability of ephrin-B2 blocking antibody (clone B11) to reverse lung fibrosis in our bleomycin-induced lung fibrosis model. In this mouse model of lung fibrosis, the C57B1/6 mouse strain develops robust lung fibrosis at day 21 post-bleomycin challenge. Equal numbers of male and female mice were used to address gender-based differences. Bleomycin (Gensia Sicor Pharmaceuticals) was administered intratracheally (i.t.) to mice by the standard method of our laboratory. A sublethal dose of 1.2 Units/k was used, which is sufficient to induce lung fibrosis without causing mortality.

In this “therapeutic strategy,” ephrin-B2 blocking antibody was administered at day 14 following bleomycin challenge, and continue for the duration of the experiment until day 21. For these experiments, the ephrin-B2 blocking antibody was injected i.v. at 4 mg/kg in 0.2 ml PBS twice per week until reaching a total dose of 20 mg/kg. Control C57B1 mice will receive control IgG2a antibody (clone C1.18.4, BioXCell). 10 mice per group were used. Timing of bleomycin and ephrin-B2 blocking antibody administration is shown in FIG. 6.

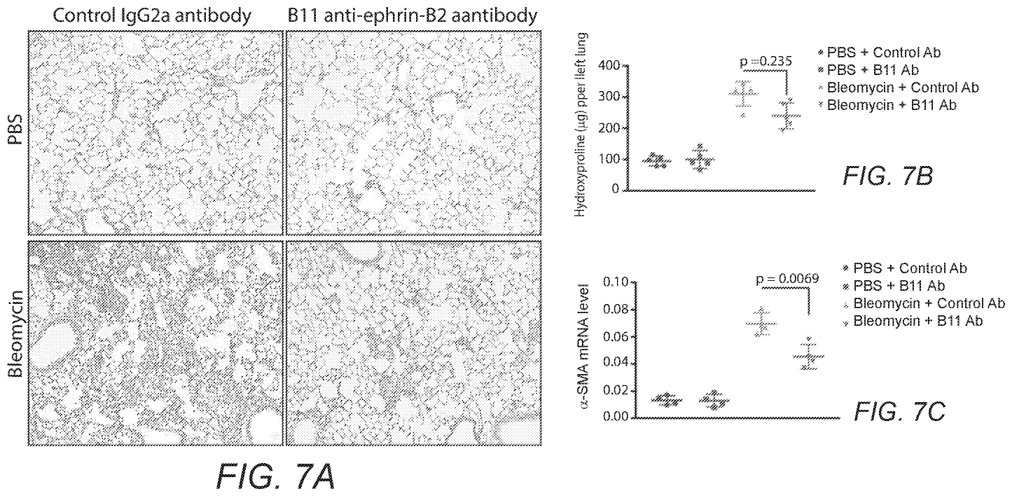

Blinded histological analysis revealed that the lung parenchymal fibrosis produced 21 d following bleomycin challenge in mice that received control IgG2a antibody was mitigated in mice treated with ephrin-B2 blocking antibody (clone B11) (FIG. 7A), and this was associated with marked reduction in lung hydroxyproline levels (FIG. 7B). To gain insight into the mechanism of action of ephrin-B2 blocking antibody in this lung fibrosis model, we assessed myofibroblast formation in vivo by qPCR. In accordance with our in vitro studies demonstrating that ephrin-B2 blocking antibody inhibits TGF-β-induced α-SMA expression in lung fibroblasts, therapeutic treatment of mice with ephrin-B2 blocking antibody reverses established lung fibrosis by downregulating expression of α-SMA, indicating that the activated cellular state of myofibroblasts in lung fibrosis is controlled by sEphrin-B2 signaling.

Statistical analyses. Differences in all other outcome measures will be tested for statistical significance by Randomized block ANOVA as described above. P<0.05 will be considered significant in all comparisons.

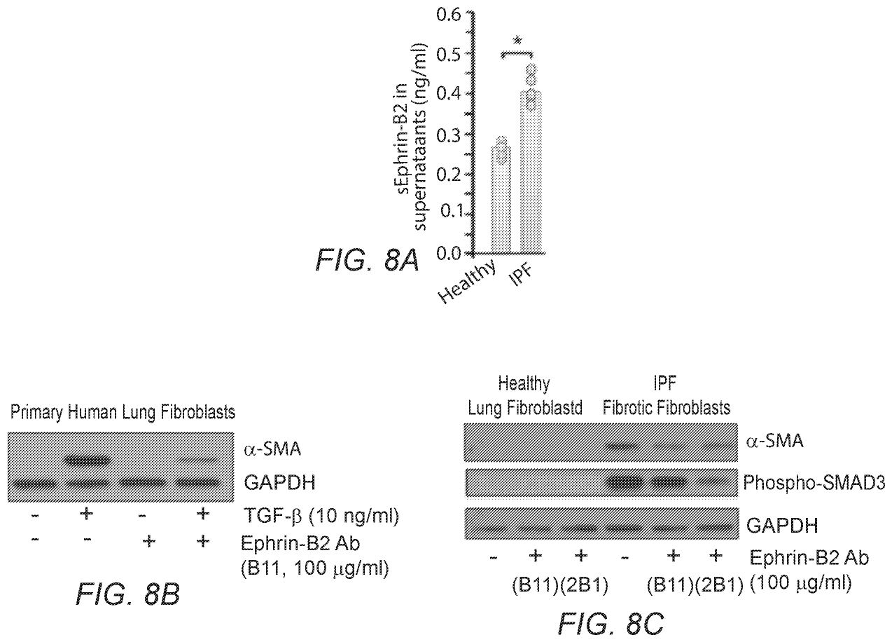

Blockade of sEphrin-B2 with neutralizing antibody reverses the activated phenotype of human lung fibroblasts from patients with IPF. To determine the relevance of our studies to human disease, we investigated the role of ADAM10-sEphrin-B2 signaling in fibroblasts isolated from the lungs of individuals with IPF and healthy controls. IPF lung fibroblasts had a substantially higher concentration of sEphrin-B2 in culture medium compared to normal lung fibroblasts in vitro (FIG. 8A), indicating that this pathway is activated in human disease and can be targeted for therapeutic intervention. To being to characterize the therapeutic potential of ephrin-B2 blocking antibodies in humans, we first examined the effect of B11 ephrin-B2 antibody on myofibroblast activation driven by TGF-β in primary human lung fibroblasts. As shown in FIG. 8B, anti-ephrin-B2 antibody prevents TGF-β-induced α-SMA protein expression in healthy primary lung fibroblasts. We next investigated whether anti-ephrin-B2 antibodies could reverse the activated phenotype of fibrotic lung fibroblasts isolated from patients with IPF. As shown in FIG. 8C, α-SMA is upregulated on IPF fibroblasts compared to control healthy fibroblasts and that treatment of IPF fibroblasts with anti-ephrin-B2 antibody (clone B11) for 48 h significantly reduces α-SMA levels (FIG. 8C), indicating that anti-ephrin-B2 therapy directly downregulates pro-fibrotic mechanisms in fibrotic fibroblasts from patients with IPF. Further, IPF fibroblasts were treated with a second anti-ephrin-B2 antibody (clone 2B1), which binds to ephrin-B2 ectodomain but does not prevent its binding to EphB4 receptor, as previously demonstrated. As shown in FIG. 8C, treatment of fibrotic IPF fibroblasts with anti-ephrin-B2 antibody (clone 2B1) similarly reduces α-SMA levels to the same extent as the clone B11. Of note, while investigating the molecular pathways modulated by anti-ephrin-B2 therapy, we found that B11 ephrin-B2 antibody did not affect increased phospho-SMAD3 levels in IPF fibroblasts compared to healthy fibroblasts, a surrogate marker of canonical TGF-β pathway. These results indicate that anti-fibrotic effects of B11 ephrin-B2 antibody do not result from modulation of canonical TGF-β pathway. Contrary, 2B1 ephrin-B2 antibody did downregulate phospho-SMAD3 levels in IPF fibroblasts, indicating that anti-fibrotic effects of this antibody results from direct modulation of the canonical TGF-β pathway. Together, both B11 and 2B1 ephrin-B2 antibodies have anti-fibrotic activity of human IPF fibroblasts albeit with mechanisms of action that appear to be distinct.

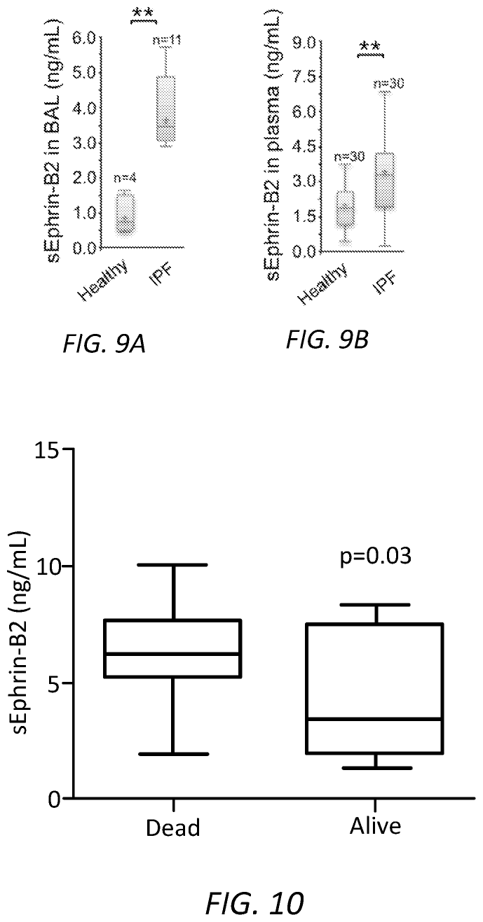

sEphrin-B2 levels are upregulated in plasma and bronchoalveolar lavage fluid in patients with IPF. The natural history of IPF is highly variable and the rate of disease progression in an individual patient is difficult to predict²⁸. Although clinical, histopathologic and radiographic analysis have been able to predict mortality in patients with IPF¹⁰, there are no clinically utilized biomarkers capable of predicting disease progression. Blood biomarkers in IPF are being investigated with the hope of improving our ability to predict disease progression²⁹⁻³². Although biomarkers of epithelial injury such as KL-6^{33,34} and endothelial activation such as VEGF³⁵ or VCAM-1^{36,37} have been found to predict poor survival in IPF, biomarkers of myofibroblast activation in IPF have been yet not identified. Our results indicate that sEphrin-B2 levels showed markedly increased concentration in the BAL fluid of 16 individuals with IPF compared to samples from 8 healthy volunteers (FIG. 9A). We then compared plasma sEphrin-B2 concentration in 30 individuals with IPF and 30 gender-matched, nonsmoking controls and observed a markedly higher concentration of plasma sEphrin-B2 in the individuals with IPF compared to the samples from the control group (FIG. 9B). Together, our data indicate that plasma sEphrin-B2 may serve as a novel myofibroblast prognostic biomarker in IPF.

Increased plasma sEphrin-B2 levels associates with increased mortality in patients with IPF. To determine the relevance of our biomarker studies to the progression of IPF, we investigated whether plasma sEphrin-B2 levels correlate with severity of illness and outcomes in IPF. As shown in FIG. 11, plasma levels in patients with IPF correlates with clinical outcomes these patients. Our data demonstrate that patients with higher baseline of plasma sEphrin-B2 levels at the time of diagnosis undergo rapid decline in lung function leading to dead of the patient.

### REFERENCES

1. Sgalla, G., et al. Idiopathic pulmonary fibrosis: pathogenesis and management. Respir Res 19, 32 (2018).

2. Selman, M., et al. Idiopathic pulmonary fibrosis: prevailing and evolving hypotheses about its pathogenesis and implications for therapy. Ann Intern Med 134, 136-151 (2001).

3. Martinez, F. J., et al. Idiopathic pulmonary fibrosis. Nat Rev Dis Primers 3, 17074 (2017).

4. Gross, T. J. & Hunninghake, G. W. Idiopathic pulmonary fibrosis. N Engl J Med 345, 517-525 (2001).

5. Lederer, D. J. & Martinez, F. J. Idiopathic Pulmonary Fibrosis. N Engl J Med 378, 1811-1823 (2018).

6. Esposito, D. B., et al. Idiopathic Pulmonary Fibrosis in United States Automated Claims. Incidence, Prevalence, and Algorithm Validation. Am J Respir Crit Care Med 192, 1200-1207 (2015).

7. King, T. E., Jr., Pardo, A. & Selman, M. Idiopathic pulmonary fibrosis. Lancet 378, 1949-1961 (2011).

8. Harari, S., Madotto, F., Caminati, A., Conti, S. & Cesana, G. Epidemiology of Idiopathic Pulmonary Fibrosis in Northern Italy. PLoS One 11, e0147072 (2016).

9. Raghu, G. Epidemiology, survival, incidence and prevalence of idiopathic pulmonary fibrosis in the USA and Canada. Eur Respir J 49(2017).

10. Collard, H. R., et al. Changes in clinical and physiologic variables predict survival in idiopathic pulmonary fibrosis. Am J Respir Crit Care Med 168, 538-542 (2003).

11. King, T. E., Jr., et al. A phase 3 trial of pirfenidone in patients with idiopathic pulmonary fibrosis. N Engl J Med 370, 2083-2092 (2014).

12. Richeldi, L., et al. Efficacy and safety of nintedanib in idiopathic pulmonary fibrosis. N Engl J Med 370, 2071-2082 (2014).

13. Wynn, T. A. Integrating mechanisms of pulmonary fibrosis. J Exp Med 208, 1339-1350 (2011).

14. Tomasek, J. J., Gabbiani, G., Hinz, B., Chaponnier, C. & Brown, R. A. Myofibroblasts and mechano-regulation of connective tissue remodelling. Nat Rev Mol Cell Biol 3, 349-363 (2002).

15. Santos, A. & Lagares, D. Matrix Stiffness: the Conductor of Organ Fibrosis. Curr Rheumatol Rep 20, 2 (2018).

16. Lagares, D., et al. ADAM10-mediated ephrin-B2 shedding promotes myofibroblast activation and organ fibrosis. Nat Med 23, 1405-1415 (2017).

17. Kullander, K. & Klein, R. Mechanisms and functions of Eph and ephrin signalling. Nat Rev Mol Cell Biol 3, 475-486 (2002).

18. Selman, M., et al. Accelerated variant of idiopathic pulmonary fibrosis: clinical behavior and gene expression pattern. PLoS One 2, e482 (2007).

19. Renzoni, E. A., et al. Gene expression profiling reveals novel TGFbeta targets in adult lung fibroblasts. Respir Res 5, 24 (2004).

20. Gardner, H., et al. Gene profiling of scleroderma skin reveals robust signatures of disease that are imperfectly reflected in the transcript profiles of explanted fibroblasts. Arthritis Rheum 54, 1961-1973 (2006).

21. Pasquale, E. B. Eph-ephrin bidirectional signaling in physiology and disease. Cell 133, 38-52 (2008).

22. Hattori, M., Osterfield, M. & Flanagan, J. G. Regulated cleavage of a contact-mediated axon repellent. Science 289, 1360-1365 (2000).

23. Tomita, T., Tanaka, S., Morohashi, Y. & Iwatsubo, T. Presenilin-dependent intramembrane cleavage of ephrin-B 1. Mol Neurodegener 1, 2 (2006).

24. Ji, Y. J., et al. EphrinB2 affects apical constriction in Xenopus embryos and is regulated by ADAM10 and flotillin-1. Nat Commun 5, 3516 (2014).

25. Lin, K. T., Sloniowski, S., Ethell, D. W. & Ethell, I. M. Ephrin-B2-induced cleavage of EphB2 receptor is mediated by matrix metalloproteinases to trigger cell repulsion. J Biol Chem 283, 28969-28979 (2008).

26. Abengozar, M. A., et al. Blocking ephrinB2 with highly specific antibodies inhibits angiogenesis, lymphangiogenesis, and tumor growth. Blood 119, 4565-4576 (2012).

27. Kwak, H., et al. Sinusoidal ephrin receptor EPHB4 controls hematopoietic progenitor cell mobilization from bone marrow. J Clin Invest 126, 4554-4568 (2016).

28. Kim, H. J., Perlman, D. & Tomic, R. Natural history of idiopathic pulmonary fibrosis. Respir Med 109, 661-670 (2015).

29. Rosas, I. O., et al. MMP1 and MMPI as potential peripheral blood biomarkers in idiopathic pulmonary fibrosis. PLoS Med 5, e93 (2008).

30. Guiot, J., Moermans, C., Henket, M., Corhay, J. L. & Louis, R. Blood Biomarkers in Idiopathic Pulmonary Fibrosis. Lung 195, 273-280 (2017).

31. Zhang, Y. & Kaminski, N. Biomarkers in idiopathic pulmonary fibrosis. Curr Opin Pulm Med 18, 441-446 (2012).

32. Tzouvelekis, A., et al. Validation of the prognostic value of MMP-7 in idiopathic pulmonary fibrosis. Respirology 22, 486-493 (2017).

33. Yokoyama, A., et al. Prognostic value of circulating KL-6 in idiopathic pulmonary fibrosis. Respirology 11, 164-168 (2006).

34. Borensztajn, K., Crestani, B. & Kolb, M. Idiopathic pulmonary fibrosis: from epithelial injury to biomarkers—insights from the bench side. Respiration 86, 441-452 (2013).

35. Ando, M., et al. Significance of serum vascular endothelial growth factor level in patients with idiopathic pulmonary fibrosis. Lung 188, 247-252 (2010).

36. Okuda, R., et al. Soluble intercellular adhesion molecule-1 for stable and acute phases of idiopathic pulmonary fibrosis. Springerplus 4, 657 (2015).

37. Shijubo, N., et al. Soluble intercellular adhesion molecule-1 (ICAM-1) in sera and bronchoalveolar lavage fluid of patients with idiopathic pulmonary fibrosis and pulmonary sarcoidosis. Clin Exp Immunol 95, 156-161 (1994).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating organ fibrosis in a subject, comprising:
identifying a subject who has organ fibrosis, and administering a therapeutically effective amount of one or more antibodies or antigen binding fragments thereof that bind to and block soluble ephrin-B2 ectodomain.

2. The method of claim 1, wherein the organ fibrosis is pulmonary, skin, kidney fibrosis, liver fibrosis or cirrhosis, systemic sclerosis, or desmoplastic tumors.

3. The method of claim 1, wherein the treatment results in a reduction in fibrosis and/or a return or approach to normal function of the organ.

4. The method of claim 1, wherein the subject has pulmonary fibrosis, and the therapeutically effective amount results in decreased lung fibrosis and improved lung function.

5. The method of claim 1, wherein the subject has pulmonary fibrosis.

6. The method of claim 5, wherein the subject has patterns of fibrosis on a chest radiograph or chest computed tomography (CT) or high-resolution CT (HRCT) scan, and bibasilar inspiratory crackles.

7. The method of claim 1, wherein the subject has systemic sclerosis.

8. The method of claim 7, wherein the subject has skin thickening of the fingers, finger tip lesions, telangiectasia, abnormal nailfold capillaries, interstitial lung disease or pulmonary arterial hypertension, Raynaud's phenomenon, and SSc-related autoantibodies.

9. The method of claim 1, wherein the subject has liver fibrosis or cirrhosis.

10. The method of claim 9, wherein the subject has fibrosis detected on biopsy or imaging.

11. The method of claim 1, wherein the antibody is a clone B11 antibody.

12. The method of claim 1, wherein the antibody is a clone 2B1 antibody.

13. The method of claim 1, wherein the antibody is a monoclonal chimeric, de-immunized or humanized antibody.

14. The method of claims 2, wherein the pulmonary fibrosis is idiopathic pulmonary fibrosis.
